(19) United States
(12) Patent Application Publication
(10) Pub. No.: US 2025/0281366 A1
(43) Pub. Date: Sep. 11, 2025

(54) SOLID COSMETIC COMPOSITION COMPRISING AT LEAST ONE CAVITY

(71) Applicant: CAPSUM, MARSEILLE (FR)

(72) Inventors: Sébastien BARDON, VEYRIER DU LAC (FR); Mathieu GOUTAYER, SAINT MALO (FR); Jean-François GONIDEC, AIX-EN-PROVENCE (FR)

(21) Appl. No.: 18/860,338

(22) PCT Filed: Apr. 26, 2023
(86) PCT No.: PCT/EP2023/060978

§ 371 Date: Oct. 25, 2024

(30) Foreign Application Priority Data
Apr. 26, 2022 (FR) 2203881

(51) Int. Cl. A61K 8/02; A61K 8/19; A61K 8/34; A61K 8/37; A61K 8/46; A61K 8/73; A61K 8/92; A61Q 19/00

(52) U.S. Cl. CPC A61K 8/0216; A61K 8/19; A61K 8/342; A61K 8/345; A61K 8/375; A61K 8/466; A61K 8/73; A61K 8/922; A61Q 19/00

(57) ABSTRACT

The present disclosure relates to a solid cosmetic composition intended for the hygiene, cleansing and/or the beautifying of a keratin material, comprising at least one cavity such that the composition has a porosity of greater than or equal to 20% by volume relative to the total volume of the solid composition, as well as to associated production methods.

The invention relates to a solid cosmetic composition intended for the hygiene, cleansing and/or the beautifying of a keratin material, more particularly the skin and/or the hair.

Hotels often offer their clients soaps and/or shampoos in the form of single-dose solid compositions. The size and the volume of the single-dose solid compositions generally fulfill the requirement of providing a good grip for the user. The size and the volume are thus generally significantly greater than same actually useful for ensuring satisfactory cleansing of the body area to be treated. A non-negligible part of the solid compositions thus ends in the trash and generates a lot of losses which, for obvious reasons, is not satisfactory from an economic and ecological point of view.

The inventors have found that it is possible to propose solid compositions overcoming the aforementioned drawback without prejudice to the good grip for the user.

### Composition

The invention thereby relates to a solid cosmetic composition intended for the hygiene, cleaning and/or the beautifying of a keratin material, comprising at least one cavity such that said composition comprises a porosity greater than 60 or equal to 20%, preferably greater than or equal to 30%, better greater than or equal to 40%, more particularly greater than or equal to 50%, more particularly greater than or equal to 70%, and most particularly greater than or equal to 80%, by volume relative to the total volume of said solid composition. The invention relates more particularly to a composition for the hygiene and/or the cleaning of a keratin material.

Thereby, a composition according to the invention may remain endowed with a size and/or a volume similar to current solid compositions but differs in the presence of at least one cavity such that the porosity of said composition is greater than or equal to 20% by volume relative to the total volume of said composition.

“Solid”, as defined by the present invention, refers to a composition which, at ambient temperature and at atmospheric pressure, is not apt to collapse under its own weight. Advantageously, one intends to refer to any composition having a compressive strength of greater than or equal to 20 g, preferably greater than or equal to 50 g, and better greater than or equal to 100 g, at ambient temperature (20-25° C.), after penetration by a cylindrical probe of revolution having a diameter of 0.8 cm into the matrix of the composition in a thickness of 1 mm at a speed of 0.5 mm/s, and removal of said probe from the matrix of the composition at a speed of 0.5 mm/s; the compression strength being measured with an analyzer such as a “LFRA Texture Analyzer” marketed by STEVENS/MECHTRIC.

A composition according to the invention is not in the form of a paste. A composition according to the invention is not a pulverulent composition, and hence is not in the form of a powder. Finally, a composition according to the invention is neither a porous solid derived from the agglomeration of powders. According to a first embodiment, a composition according to the invention is not a puffed composition, and hence is not a foam. According to a second embodiment, a composition according to the invention may be a puffed composition, and hence may be in the form of a foam.

Preferably, a solid composition according to the invention is not coated with a water-soluble coating.

“Porosity”, as defined by the present invention, refers to all the voids of a solid material, the voids being able to be filled with fluids, in particular water, when the solid composition is brought into contact with an aqueous solution, e.g. water such as tap water, shower or bath water.

“Cavity”, as defined by the present invention, refers to an empty space inside a solid body and which communicates directly with the external medium, like e.g. the cavities illustrated in FIGS. 1 to 3 and 6. A cavity can thus be referred to, without distinction, by the terms “orifice”, “pore” or “hole”. Advantageously, the cavity(ies) is/are through, i.e. the cavity designates an empty space inside a solid body and which communicates directly with the external medium on both sides of the solid body.

Thereby, the volume of the cavity(ies) in a solid composition according to the invention represents at least 20%, more particularly at least 30%, preferably at least 40%, more particularly at least 50%, even at least 60%, more particularly at least 70%, and most particularly at least 80%, of the total volume of said solid composition.

Advantageously, a solid composition according to the invention has a density comprised between 0.2 and 0.8, preferably between 20 and 0.7, more particularly between 20 and 0.6.

A solid composition according to the invention may have a total volume comprised between 0.125 cm³ and 100 cm^{3,} preferably between 0.250 cm³ and 75 cm³, more particularly between 0.5 cm³ and 50 cm³, better between 1 cm³ and 25 cm³, and most particularly between 2.5 cm³ and 15 cm³.

A solid composition according to the invention may have a weight comprised between 0.025 g and 200 g, preferably between 0.25 g and 150 g, more particularly between 0.5 g and 100 g, better between 1 g and 75 g, most particularly between 2.5 g and 50 g, even between 5 g and 25 g (“g” corresponding to the “gram” unit).

Advantageously, a solid composition according to the invention may have a weight comprised between 0.25 g and 200 g, preferably between 0.50 g and 150 g, more particularly between 1 g and 100 g, better between 2.5 g and 75 g, and most particularly between 5 g and 50 g (“g” corresponding to the “gram” unit).

According to a first preferred embodiment, a solid composition according to the invention comprises a ratio between the volume of the cavities and the total volume of the composition comprised between 0.05 and 0.9, preferably between 0.1 and 0.8, more particularly between 0.2 and 0.9, preferably between 0.2 and 0.8, better between 0.2 and 0.7, even between 0.2 and 0.6.

“Total volume” refers to the volume resulting from the sum of the volume of solid and the of the volume of the cavities.

Within the framework of the present invention, the cavities may be considered to be macroporosity, i.e. pores the minimum width, the diameter even, of which is greater than or equal to 0.5 mm, preferably greater than or equal to 0.75 mm, and more particularly greater than or equal to 1 mm, even comprised between 0.5 mm et 2 cm, most particularly between 1 mm et 2 cm, preferably between 2 mm et 1 cm, and more particularly between 3 mm et 75 mm, better between 4 mm et 50 mm, and most particularly between 5 mm et 25 mm. More particularly, the pores have a minimum width, even a diameter, comprised between 0.5 mm et 5 mm, preferably between 1 mm and 3 mm.

As such, the Applicant indicates that, at equivalent porosity, manufacturing a stable solid composition is:
easy in the presence of small cavities, e.g. the cavities present in solid substrates where the porosity is consecutive to the addition of a gas during the manufacturing process, as described in U.S. Pat. No. 8,349,341, but
much more difficult in the presence of macroporosities such as same considered in the present invention.

The presence of cavities in the contents and/or volumes and/or dimensions considered hereinabove makes it possible to obtain solid compositions which require less raw materials while remaining endowed with satisfactory gripping properties. A solid composition according to the invention is thus able to generate no loss or less loss. Such weight saving further has the advantage of permitting less polluting and less expensive transport. A solid composition according to the invention is thus both ecologically and economically advantageous.

Moreover, the inventors observed an unexpected effect in terms of texture and transformative effect when a solid composition according to the invention is placed in the presence of an aqueous phase. Indeed, the inventors have observed that a solid composition according to the invention, in contact with an aqueous phase, produces a greater quantity of foam and more rapidly than with a solid composition of the same size and volume but without a cavity, even shows a more rapid transformation kinetics between the original solid state and a liquid state, e.g. such as a gel or a cream. Without wishing to be bound by any theory, the inventors believe that the presence of cavity(ies) within the solid composition makes it possible to facilitate the transition to a viscous liquid composition, e.g. when the solid composition is mixed with the hands, or leads to an increase in the contact surface area with the aqueous phase, more particularly water, and thus a better hydration of said solid composition.

Thereby, the invention even makes it possible to regulate the speed at which the solid composition will foam, will swell and/or melt, by adjusting the developed surface to constant volume, by regulating the percentage of porosity, and more particularly by acting on the shape and/or the number and/or dimensions of the cavities, even the number of walls and/or the thickness of the walls of the cavities.

Advantageously, the size and/or the volume of the cavities can be adjusted to store and/or react with the right amount of water necessary to achieve a final liquid composition endowed with satisfactory/expected properties, even optimized and/or homogeneous (i.e. between different single doses), e.g. in terms of viscosity, hydration, ease of spreading, the amount of foam formed and/or transformative effect.

Thereby, a composition according to the invention even makes it possible to obtain evanescent compositions, namely compositions apt to be transformed into liquid compositions, more particularly in the form of gels or creams, in contact with an adequate quantity of water.

Finally, the advantages described hereinabove in terms of the ability to foam and/or transform into a liquid composition may also require a lower consumption of water, which is an additional advantage from an ecological and economic point of view.
The solid character of a composition according to the invention may be based on a phase consisting mainly, or even entirely, of a fatty phase comprising at least one lipophilic gelling agent (also referred to hereinafter as “solid fatty phase”).

Also, the solid character of a composition according to the invention may be based on a phase derived from a saponification reaction (or “pasting”), and thus result from the transformation of a fatty phase comprising at least one oil, preferably a vegetable oil, under the action of soda and, where appropriate, heat (also referred to hereinafter as “soap phase”).

Also, the solid character of a composition according to the invention may be based on a phase comprising at least one synthetic detergent (or surfactant) (also referred to hereinafter as “SYNDET phase”). As an illustration, it is CETYL ALCOHOL, in whole or in part, which contributes to imparting to SYNDET the solid character thereof.

The solid character of a composition according to the invention can thus be based on (i) a solid fatty phase, (ii) a soap phase and/or (iii) a SYNDET phase. Hereinafter in the description, the “solid fatty phase”, the “soap phase” and the “SYNDET phase” may commonly be referred to by the expression “solid phase”.

Thereby, a composition according to the invention comprises at least one solid phase.

Preferably, a composition according to the invention is single-phase, and hence consisting only of the solid phase.

According to a particular embodiment, a composition according to the invention may also comprise an aqueous phase dispersed in the solid phase, in particular as defined hereinbelow.

According to a particular embodiment, a composition according to the invention may also comprise an aqueous phase dispersed in the solid composition, preferably in encapsulated form.

Preferably, a composition according to the invention is a single-dose composition.

Such embodiment is particularly advantageous from a hygienic point of view in that same prevents repeated contacts between the solid composition and the surface to be treated.

Thereby, the invention further relates to a packaging comprising a plurality of solid compositions according to the invention in the form of single-doses, e.g. different fragrance, shape, color and/or cosmetic active ingredient/effect.

Preferably, a composition according to the invention is a topical composition. A composition according to the invention is not an oral composition.

The invention thereby relates to a solid cosmetic composition intended for the hygiene, cleaning and/or the beautifying of a keratin material.

A solid composition according to the invention may have any shape, e.g. a spherical, ellipsoid, tetrahedral or polygonal shape, more particularly in the shape of a parallelogram, preferably a rectangular parallelogram.

Preferably, in particular to provide a good grip, a solid composition according to the invention has:
a width, or even a diameter, comprised between 1 cm and 10 cm, preferably between 2 cm and 8 cm, more particularly between 3 cm and 6 cm, and better between 4 cm and 5cm; and/or
a height (or thickness) of between 0.5 cm and 5 cm, preferably between 1 cm and 4 cm, and more particularly between 2 cm and 3 cm.

The cavity(ies) of a solid composition according to the invention can have any shape, e.g. a round, oblong, ovoid, triangular, polygonal, more particularly hexagonal, honeycomb shape, or in the shape of a paving (or cutout), e.g. of such as a Voronoï diagram, preferably in the shape of a honeycomb, as illustrated in FIGS. 3 and 6.

The cavity(ies) of a solid composition according to the invention can also represent letters or the logo of a brand or a company, as illustrated in FIG. 2.

A solid composition according to the invention comprises one or a plurality of cavities, preferably a plurality of cavities, either identical or different.

According to a first variant, the composition according to the invention comprises at least one cavity, more particularly a through cavity.

According to a second preferred variant, a composition according to the invention comprises a plurality of cavities, more particularly through cavities, as illustrated in FIGS. 3 and 6.

The cavities are through cavities, advantageously along an axis x-x′.

According to a particular embodiment, a composition according to the invention may comprise at least two cavities connected together. Such an embodiment is advantageous because same enhances capillarity phenomena when brought into contact with water, which improves the aforementioned technical effects.

Advantageously, a solid composition according to the invention comprises a plurality of cavities which take a honeycomb-shaped structure, as illustrated in FIG. 3. The honeycomb shape is particularly advantageous in that same permits the manufacture of a solid composition provided with a very advantageous “volume of the cavity(ies)/total volume of the composition” ratio, more particularly greater than or equal to 0.5, even greater than or equal to 0.75, without prejudice to the grip and the robustness of said composition to the mechanical stresses likely to be applied thereto and/or to storage. In other words, the honeycomb structure is an excellent compromise between cavity volume and mechanical strength.

Advantageously, the cavity(ies) of a solid composition according to the invention comprise(s) walls having a thickness comprised between 10 μm and 1,500 μm, preferably between 20 μm and 1,000 μm, more particularly between 30 μm and 750 μm, most particularly between 50 μm and 500 μm, even between 100 μm and 400 μm. More particularly, the cavity(ies) of a solid composition according to the invention comprises/comprise walls having a thickness comprised between 250 μm and 500 μm.

Advantageously, a solid composition according to the invention comprises a plurality of cavities which take a honeycomb-shaped structure comprising walls having a thickness comprised between 10 μm and 600 μm, preferably between 20 μm and 500 μm, more particularly between 30 μm and 400 μm, most particularly between 50 μm and 300 μm, even between 100 μm and 200 μm.

According to a particular embodiment, a solid composition according to the invention, in particular when same takes a honeycomb configuration as described hereinabove, may comprise a plurality of sections, more particularly polygonal, each of the sections defining a breakable perimeter permitting the separation of at least one section from the remainder of the solid composition. Thereby, according to such embodiment, each section of the solid composition corresponds to a single dose.

According to a particular embodiment, a composition according to the invention may also comprise additional patterns distinct from the cavities, said patterns being able, e.g., to be produced by embossing.

A person skilled in the art would make sure to choose the possible constituent compound(s) of a composition according to the invention and/or the amount thereof so that the advantageous properties of a composition according to the invention, are not altered or not substantially altered. Also, a person skilled in the art would make sure to choose the nature and/or the amount of additional compound(s) depending on the solid character, even anhydrous when considered, of said composition and/or manufacturing method. Such adjustments fall within the general knowledge of a person skilled in the art.

In particular, a composition according to the invention advantageously comprises at least one lipophilic gelling agent, more particularly at least one solid fatty substance, preferably chosen from at least one wax, at least one pasty fatty substance, at least one butter, and mixtures thereof, e.g. as described in WO2021/234135.

Advantageously, a lipophilic gelling agent is a thermosensitive gelling agent, i.e. one that reacts to heat, and in particular is a gelling agent that is solid at room temperature and liquid at a temperature above 50° C., preferably above 60° C., and more preferably above 70° C. Preferably, a heat-sensitive lipophilic gelling agent according to the invention has a melting point between 50° C. and 130° C., and preferably between 60° C. and 120° C.

In particular, a composition according to the invention may comprise from 10% to 90%, preferably from 20% to 80%, in particular from 30% to 70%, and better still from 40% to 60%, by weight of lipophilic gelling agent(s) relative to the total weight of the composition.

A composition according to the invention may comprise at least one unsaturated, monounsaturated or polyunsaturated fatty acid, preferably C₆-C₂₂ fatty acid, more particularly C₈-C₂₀ fatty acid, and better C₁₂-C₁₈ fatty acid, such as, e.g., lauric acid (C₁₂), myristic acid (C₁₄), palmitic acid (C₁₆), stearic acid (C18), ricinoleic acid (C₁₈), and mixtures thereof.

A composition according to the invention may comprise at least one oil, more particularly a vegetable oil, such as, e.g., coconut oil, olive oil, sweet almond oil, castor oil, Babassu oil, and mixtures thereof. Examples of such oils are those described in WO2021/234135.

In particular, a composition according to the invention may comprise from 10% to 90%, preferably from 20% to 80%, in particular from 30% to 70%, and better still from 40% to 60%, by weight of oil(s) relative to the total weight of the composition.

A composition according to the invention may comprise at least one detergent agent, e.g. a surfactant, chosen in particular from sodium cocoyl isethionate, sulphosuccinates, alpha oleficategories, alkyl glyceryl ether sulfonate, sodium cocoyl monoglyceride sulfate, betaine, and mixtures thereof. Such is in particular the case when the composition according to the invention is a SYNDET (which derives from the term “synthetic detergents”) also referred to as “soap without soap” or “dermatological bar”.

A composition according to the invention may comprise at least one lipophilic gelling agent, preferably heat-sensitive, at least one oil and/or at least one detergent agent, and mixtures thereof.

According to a first variant, a composition according to the invention is anhydrous. “Anhydrous”, as defined by the present invention, refers to a composition which, before any contact with water, comprises a water content of less than 5% by weight, preferably less than 1% by weight and more particularly less than 0.5% by weight, relative to the total weight of said composition. Such is in particular the case when a composition according to the invention is a SYNDET.

According to a second variant, a composition according to the invention comprises a continuous fatty phase and a dispersed aqueous phase. Such is in particular the case when a composition according to the invention is a soap or when the oily (or fatty) phase additionally comprises at least one dispersed aqueous phase, e.g. in encapsulated form such as e.g. in the form of matrix spheres (or beads or small balls) or such as cores/shells (or capsules) e.g. in the form of capsules as described in WO2010/063937.

Thereby, a solid composition according to the invention may also comprise at least one aqueous phase dispersed in the solid composition, preferably in encapsulated form.

According to one embodiment, the mass percentage of water of the aqueous phase may then be comprised between 0.5% and 50%, preferably between 1%, and 40%, more particularly between 2.5% and 30%, and better between 5% and 20%, by weight relative to the total weight of the composition.

A composition according to the invention, more particularly the fatty phase, even the dispersed aqueous phase when present, may further comprise at least one additional compound different from the aforementioned lipophilic gelling agent, oils and/or detergent agents.

A composition according to the invention may also comprise powders; flakes; coloring agents, in particular chosen from water-soluble or water-insoluble, liposoluble or non-liposoluble, organic or inorganic coloring agents, materials with an optical effect, liquid crystals, and mixtures thereof; particulate agents insoluble in the fatty phase; scenting agents; organic or inorganic fillers, in particular pigments; preservatives; humectants; effervescent agents; disintegrants (e.g. Croscarmellose, carboxymethylcellulose, etc.); thinners; disintegration-promoting agents; stabilizers; chelating agents; softeners; modifying agents chosen amongst texture agents (or gelling agents) and more particularly hydrophilic gelling agents, agents modifying the pH, the osmotic force and/or modifiers of the refractive index etc. . . . or any usual cosmetic additive; and mixtures thereof.

Advantageously, a composition according to the invention also comprises at least one effervescent agent and/or at least one disintegrant.

Preferentially, the composition comprises between 0.5% and 50%, more particularly between 1% and 40%, and better between 5% and 30%, by weight of disintegrant relative to the total weight of the composition.

A disintegrating agent may be chosen from sodium starch glycosates; starch, more particularly wheat starch or maize starch; pregelatinized or partially pregelatinized starches; sodium carboxymethyl starch, weakly substituted, where appropriate; starches composed of lactose starch particles and maize starch particles; starches composed of starch and peas; starches composed of starch and potato; starches composed of starch and wheat; disintegrating agents containing mannitol; crystalline cellulose; microcrystalline cellulose or mixture of microcrystalline cellulose with gum xanthan; sodium carboxymethyl cellulose; croscarmellose calcium; croscarmellose sodium; ethylcellulose; substituted hydroxypropyl cellulose; hydroxypropylmethyl cellulose; cross-linked polyvinylpyrrolidone; cross-linked polyacrylates, alginic acid and derivatives thereof, e.g. sodium alginate; and mixtures thereof.

Preferably, a composition according to the invention may also comprise at least one diluent, preferably chosen from the group consisting of microcrystalline cellulose, mannitol, lactose, starch, sodium carbonate, sodium bicarbonate and calcium carbonate, and mixtures thereof. Preferably, the composition comprises a content of less than 8%, even less than 5%, by weight of diluent(s) relative to the total weight of the solid composition.

Preferably, a composition according to the invention may further comprise at least one disintegration promoter, preferably calcium silicate.

A composition according to the invention, more particularly the fatty phase, even the dispersed aqueous phase when present, may further comprise at least one biological/cosmetic active agent chosen from moisturizing agents, vitamins, cicatrizing agents, depigmenting agents, UV filters, desquamating agents, antioxidants, active agents stimulating the synthesis of dermal and/or epidermal macromolecules, skin relaxant agents, antiperspirants, soothing agents and/or anti-aging agents, and mixtures thereof.

As hydrophilic gelling agents, mention may be made of same described in FR3041251.

A composition according to the invention may further comprise at least sodium lactate and/or at least one salt. Such ingredients have in particular the advantage of hardening the soap.

A composition according to the invention may also comprise at least one solvent apt to evaporate, completely or partially, during the manufacturing process, as described hereinafter. As solvent, mention may be made e.g., of an apolar or slightly polar organic solvent, e.g. cyclohexane, THF (tetrahydrofuran), and mixtures thereof.

Of course, a person skilled in the art would make sure to choose any additional compound(s) and/or the amount thereof so that the advantageous properties of the composition according to the invention, are not altered or not substantially altered by the envisaged admixture. Also, a person skilled in the art would make sure to choose the nature and/or the amount of additional compound(s) depending on the aqueous or fatty nature of the phase considered and/or with regard to the method of manufacturing the composition. Such adjustments fall within the general knowledge of a person skilled in the art.

When the composition according to the invention is a soap and/or a shampoo, said composition advantageously satisfies at least one of the criteria (or indices) among same defined hereinafter:
the composition has a cleansing power between 12 and 22, the highest number meaning that the soap is more cleansing;
the composition has a hardness index comprised between 29 and 54, the highest number indicating a hardest soap and the lowest number indicating a milder soap;
the composition has an Emollience index comprised between 44 and 69;
the composition has a foaming power comprised between 14 and 46, the highest number indicating larger bubbles and the lowest number indicating smaller bubbles;
the composition has a smoothness index comprised between 16 and 48;
the composition has an iodine value comprised between 41 and 70; and/or
the composition has an INS (lodine Number Saponification) comprised between 136 and 170, preferably between 150 and 165.

The above criteria (or indices), as well as the method of measurement thereof, fall within the general knowledge of a person skilled in the art.

The solid character of a composition according to the invention advantageously makes it possible to dispense with conventional packaging. Indeed, the packaging of a solid composition according to the invention may be limited to a simple packaging in a paper.

Advantageously, a solid composition according to the invention does not consist of a fiber lattice.

Preferably, a solid composition according to the invention does not comprise polyvinyl alcohol (PVA or PVOH), polyacrylate, copolymer of acrylic acid and methacrylic acid, polyvinylidene fluoride (PVDF), polymethyl methacrylate (PMMA), polyvinylpyrrolidone, polyalkylene oxide, polyacrylonitrile (PES or PAN), polymethacrylate of polyethylene terephthalate, polyethylene, polypropylene, nylon, hyaluronic acid, starch and derivatives thereof, pullulan, gelatin, cellulose (e.g. rayon), hydroxypropyl methylcellulose, methylcellulose, carboxymethyl cellulose, cotton, and one of the mixtures thereof.

A composition according to the invention does not comprise (or does not rest on) a water-soluble solid support, e.g. such as a hydrogel, more particularly based on starch, where appropriate destructured, and/or poloxamer 407.

### Manufacturing Method

A composition according to the invention is obtained by any method known to a person skilled in the art, suitable for the manufacture of a solid composition, provided the adaptations needed to ensure the formation of the cavities and thus to satisfy the required criterion of porosity.

Preferably, the method of manufacturing a solid composition according to the invention does not comprise a step of aeration/injection of a gas, e.g. air, where appropriate followed by a step of forming and then drying.

### Method No. 1

According to a first variant, a method of manufacturing a composition according to the invention comprises at least the steps consisting in:

(i) having available a composition in liquid or semi-liquid form;

(ii) casting the composition in a mold and drying until a solid composition is obtained,

(iii) cutting the solid composition into the desired shape, and

(iv) forming cavities to obtain a solid composition according to the invention.

More particularly, such method of manufacturing a composition according to the invention comprises at least the steps consisting of:

(i) having available a fatty phase, a phase derived from a saponification reaction and/or a phase comprising at least one synthetic detergent (or surfactant) (i.e. SYNDET phase), in liquid or semi-liquid form;

(ii) casting the phase of step (i) into a mold and drying until reaching a solid phase,

(iii) cutting the solid phase the desired shape, and

(iv) forming cavities, whereby a composition according to the invention is obtained.

According to a particular embodiment, such a manufacturing method is a saponification method (or “pasting).

Step (i) is advantageously carried out under hot conditions.

Advantageously, steps (iii) and (iv) are simultaneous.

Advantageously, steps (ii), (iii) and (iv) are simultaneous, in which case the mold has a shape suitable for ensuring the formation of the cavity(ies).

Step (iv) is advantageously carried out by means of a clay cutter.

Method No. 1 is advantageous in that the waste obtained at the end of step (iv) can be recovered during a subsequent manufacture. Indeed, the off-cuts can be heated again and added again in a subsequent method No. 1 at step (i) and/or (ii).

Advantageously, in particular when the composition according to the invention is a soap and/or a shampoo, a method according to the invention may further comprise, before step (ii), at least one washing step, in order to eliminate the remaining soda.

### Method No. 2

According to a second variant, a method of manufacturing a composition according to the invention is an extrusion method.

Such a manufacturing method according to the invention comprises at least the steps consisting of:

(a) having a composition in liquid or semi-liquid form;

(b) passing the composition of step (a) through an extrusion device equipped with a die (or extrusion grating) with a shape suitable for forming a solid bar, and

(c) cutting the bar obtained in step (b) to obtain a solid composition according to the invention.

More particularly, such method of manufacturing a composition according to the invention comprises at least the steps consisting of:

(a) having available a fatty phase, a phase derived from a saponification reaction and/or a phase comprising at least one synthetic detergent (or surfactant) (i.e. SYNDET phase), in liquid or semi-liquid form;

(b) passing the phase of step (a) through an extrusion device equipped with a die (or extrusion grating) with a shape suitable for forming a solid bar, and

(c) cutting the solid bar obtained in step (b), thereby obtaining a composition according to the invention.

For obvious reasons, step (b) is carried out at a temperature suitable for obtaining a composition with sufficient viscosity and apt to keep the shape given by the die.

For obvious reasons, the shape of the die of the extrusion device is also suitable for the formation of the cavities.

Advantageously, step (b) is carried out at a temperature lower than the ambient temperature, and e.g. at a temperature lower than or equal to 15° C., or even lower than or equal to 10° C.

In step (b), the extrusion device comprises a die having a shape suitable for forming at the solid bar the cavity(ies) and the desired level of porosity.

As an illustration, an extrusion method according to the invention comprises a die such as the die illustrated in FIG. 4.

Step (c) can be carried out using a knife or a wire, such as e.g. a butter cutting wire.

Preferably, steps (b) and (c) are simultaneous.

Preferably, the method of manufacturing a composition according to the invention is the extrusion method No. 2 described hereinabove.

According to a particular embodiment, applicable to methods No. 1 and No. 2 described above, step (i)/(a) consists in having a fluid composition which may comprise at least one solvent apt to evaporate, completely or partially, during step (ii)/(b), or even in addition during step (iii) and/or (iv)/(c).

As solvent, mention may be made e.g., of an apolar or slightly polar organic solvent, e.g. cyclohexane, THF (tetrahydrofuran), and a mixture thereof.

In the above methods No. 1 and No. 2, when the composition comprises at least one fatty phase comprising at least one lipophilic gelling agent, step (i)/(a) may require heating said composition to a temperature higher than the highest melting point of the lipophilic gelling agents present in said composition.

According to a particular embodiment, the extrusion method No. 2 can be carried out by a 3D printing technique.

### Use

A composition according to the invention is intended for the hygiene and/or the beautifying of a keratin material, more particularly the skin and/or the hair.

More particularly, a composition according to the invention is intended for the hygiene of a keratin material, more particularly of the skin and/or the hair, such as e.g. toothpastes, deodorants, shower gels, intimate cleansing gels, soaps, shampoos, mouthwashes, hair color products (hair dyes and bleaches), hair care products, cleansing products, shaving and depilatory products, such as aftershaves, depilatory creams and shaving creams, preparations for bath and shower such as e.g. bath foams, bath oils and bath salts. Preferably, a solid composition according to the invention is a soap and/or a shampoo.

Advantageously, a solid composition according to the invention is not in the form of a mask.

Advantageously, a solid composition according to the invention is intended to be brought into contact with an aqueous phase, more particularly with water, before, simultaneously and/or after the application thereof to the keratin material to be treated.

Thereby, according to a first embodiment, more particularly when a composition according to the invention is of the single-dose type, bringing in contact with water is extemporaneous, i.e. just before use and/or simultaneously if applied to a wet keratin material.

According to a second embodiment, when a composition according to the invention is not of the single-dose type, the bringing in contact with water is carried out during the first use only, and presupposes a packaging of the “solid composition/water” mixture in a suitable container. For obvious reasons, thereof requires a solid composition of suitable formula and a sufficient quantity of water to ensure the transition from a solid composition to a liquid composition, e.g. in gel form. Thereof also requires the presence in the solid composition of at least one preservative in order to guarantee good preservation over time of the liquid composition thereby formed.

Finally, the present invention also relates to a non-therapeutic cosmetic treatment method for hygiene and the beautifying of a keratin material, more particularly of the skin and/or the hair, comprising at least one step of applying to said keratin material of at least one composition as defined hereinabove.

### BRIEF DESCRIPTION OF FIGURES

FIG. 1 shows a composition according to the invention, with a parallelepiped shape having a cavity of cylindrical shape. A is the side view and B is the top view.

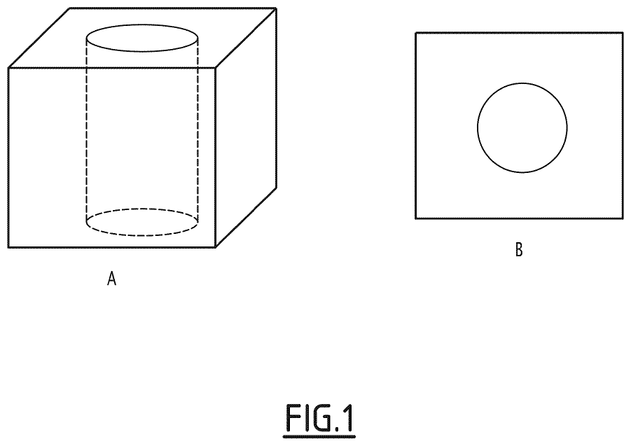

FIG. 2 represents an ovoid-shaped composition according to the invention with cavities (top view), representing the word “SOAP”.

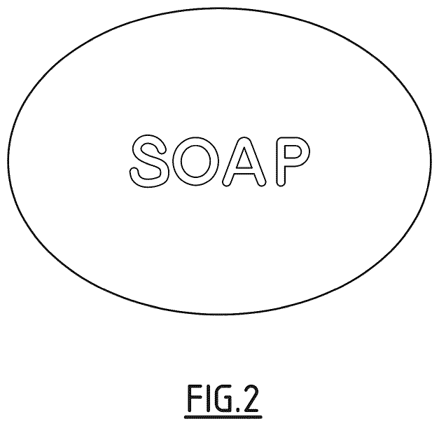

FIG. 3 shows a composition according to the invention with honeycomb cavities (top view).

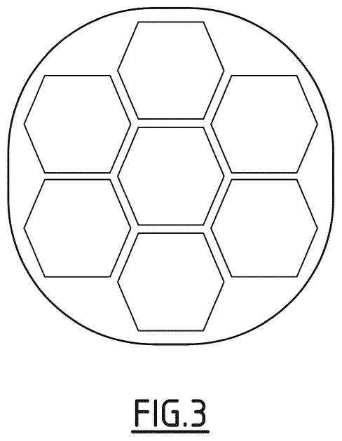

FIG. 4 shows a die used for the method of the invention.

FIG. 5 shows a comparative die.

FIG. 6 shows other examples of solid compositions according to the invention.

### EXAMPLES

Using a mixer and a water bath at 80° C., the raw materials are mixed in the order and quantities described in the following table until a homogeneous mixture is obtained

| | Weight | |
|---|---|---|
| Name | (kg) | Phase |
| Water | 85 | A |
| Pure caustic soda | 35 | A |
| Coconut oil | 80 | B |
| Shea butter | 60 | B |
| Olive oil | 100 | B |
| Sweet almond oil | 8 | C |
| Total | 368 | |

Step 1—manufacturing:

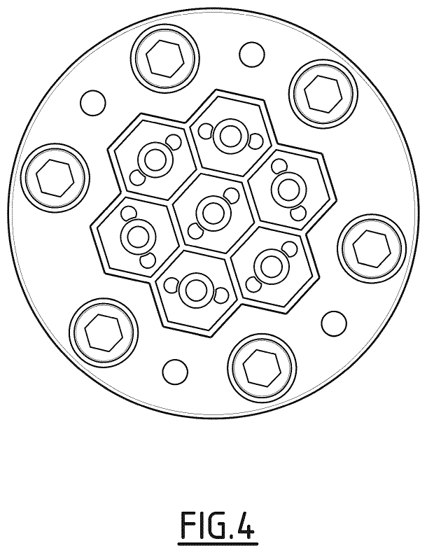

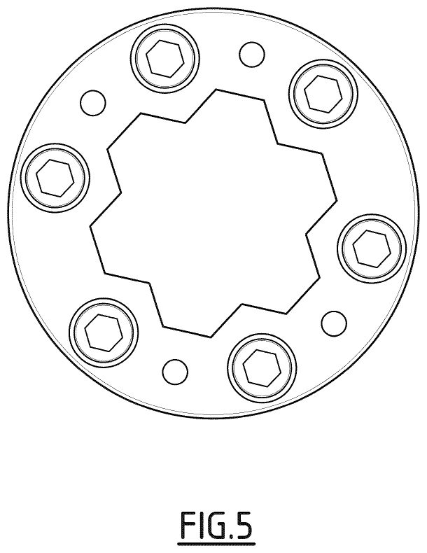

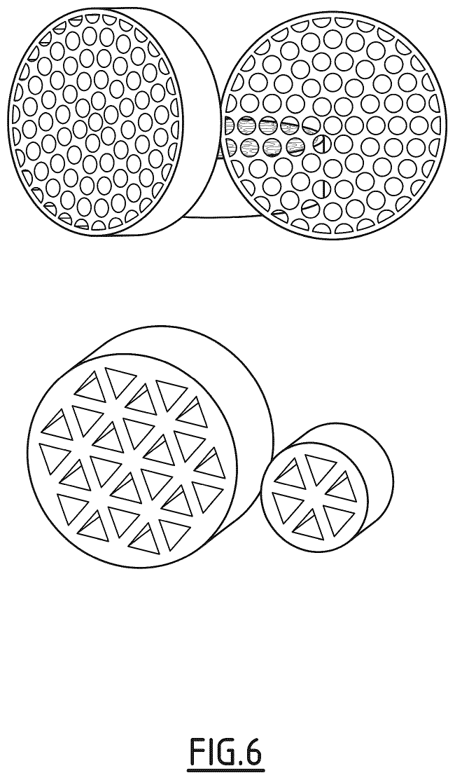

In a first container, water and caustic soda are mixed (=mixture A);
in a second container, the coconut oil and shea butter are melted and mixed in a water bath (70° C.), then the olive oil is added and mixed until reaching homogenization (=mixture B);
When the temperature of mixtures A and B is below 40° C., mixture A is poured into mixture B and sweet almond oil is added, then mixed until reaching homogenization (=mixture C), and
Mixture C is left to rest for 48 hours at ambient temperature. The pH of the composition is between 9 et 10.
   Step 2—extrusion:
The mixture C obtained in step 1 is injected into an extruder provided, in a first case, with a die according to the invention as illustrated in FIG. 4 and, in a second case, with a comparative die as illustrated in FIG. 5;
At the outlet of the die, the respectively extruded soap bars are cut so as to obtain the weights and volumes described in Table 1 below.
Single-dose soaps are obtained in the form of a solid composition according to the invention with a honeycomb structure (soap 1, like e.g. same described in FIG. 6) and a comparative solid composition with a general shape identical to soap 1 but with no cavity (soaps 2 and 3), respectively.

| | Soap 1 | Soap 2 | Soap 3 |
|---|---|---|---|
| | (invention) | (comparative) | (comparative) |
| Weight (mg) | 250 | 250 | 1000 |
| Volume (in | 1 | 0.25 | 1 |
| cm³) | | | |
| Porosity (%) | >75 | 0 | 0 |

Step 3—application:

Step 3 is carried out on a panel of 18 women and 18 men between the ages of 22 and 47 (hereinafter the “candidates”), 3 groups being then formed, each group being formed of 6 women and 6 men (groups A, B and C).
For each group,
3.1/The candidates pour a glass filled with 30 cl of tap water on both their hands.
3.2/Then, candidates wash their hands with soap 1, 2 or 3.
3.3/The candidates rinse their hands with tap water and then dry their hands.

Each of groups A, B and C will use soaps 1, 2 and 3, but each in a different order.

During step 3, the candidates assess the performance of the soaps 1, 2 and 3 in terms of (I) gripping capacity, (ii) foaming capacity and (iii) state of the soap after use.

Scoring criteria:

| SCORING | | | |
|---|---|---|---|
| CRITERIA | 0 | 1 | 2 |
| Grip | Satisfactory | Average | Poor |
| Foam | High | Average | Insufficient |
| Condition of | Total | Some | General |
| the soap after | disappearance | residue | structure kept |
| use | | | |

The means of the results obtained with the three groups A, B and C are presented in the following table, bearing in mind that the observed trends are identical between the three groups.

| | Soap 1 | Soap 2 | Soap 3 |
|---|---|---|---|
| | (Invention) | (Comparative) | (Comparative) |
| Grip | 0 | 2 | 0 |
| Foam | 0 | 1 | 1 |
| Condition after use | 0 | 0 | 2 |

With classic soaps 2 and 3, it is observed that when the size is too small, the grip is poor and handling is difficult and when the product is too bulky, it is not possible to consume same completely during a single-dose use, which generates a lot of losses.

The soap according to the invention 1 makes it possible to combine the advantages of classic soaps 2 and 3 without the drawbacks thereof.

A composition according to the invention, due to the presence of cavities in the contents considered, makes it possible to obtain solid compositions able to generate more foam with less material while generating less loss, and without prejudice to the grip-related properties.

A solid composition according to the invention thus consumes less raw materials and is thus both ecologically and economically advantageous.

A study similar to the study described hereinabove was also carried out with a SYNDET (also referred to as “soap without soap” or “dermatological bar”), described in the table hereinbelow. Identical results were obtained.

| | Weight |
|---|---|
| Raw material | (in %) |
| GLYCERINE | QSP* |
| SODIUM COCOYL ISETHIONATE | 40 |
| CROSCARMELLOSE SODIQUE | 4 |
| CETYL ALCOHOL | 13 |
| POLYGLYCERYL 4 LAURATE | 2 |
| Total | 100 |

*Quantum satis

1. A solid cosmetic composition intended for the hygiene, cleaning and/or the beautifying of a keratin material comprising at least one cavity such that the said composition comprises a porosity greater than or equal to 20% by volume relative to the total volume of said solid composition.

2. The solid composition according to claim 1, wherein the porosity is greater than or equal to 30%, by volume relative to the total volume of said solid composition.

3. The solid composition according to claim 1, wherein the cavity(ies) is/are through.

4. The solid composition according to claim 1, said composition comprising one or a plurality of cavities, either identical or different.

5. The solid composition according to claim 1, wherein the cavity(ies) is/are round, oblong, ovoid, triangular, polygonal, honeycomb, or in the shape of a paving (or cutout).

6. The solid composition according to claim 1, wherein the ratio between the volume of the cavity(ies) and the total volume of the composition is comprised between 0.2 and 0.9.

7. The solid composition according to claim 1, wherein said composition comprises at least one lipophilic gelling agent, at least one oil, and/or at least one detergent, and mixtures thereof.

8. The solid composition according to claim 1, wherein said composition further comprises at least one aqueous phase dispersed in the solid composition.

9. The solid composition according to claim 1, wherein said composition is a single-dose composition.

10. The solid composition according to any one of the preceding claim 1, wherein said composition is a topical composition.

11. A method for manufacturing a the solid composition according to claim 1, comprising at least the steps consisting of:
(i) having available a fatty phase, a phase derived from a saponification reaction and/or a phase comprising at least one synthetic detergent (or surfactant) (i.e. SYNDET phase), in liquid or semi-liquid form;
(ii) casting the phase of step (i) into a mold and drying until reaching a solid phase,
(iii) cutting the solid phase to the desired shape, and
(iv) forming cavities whereby the solid composition is obtained.

12. The method for manufacturing the solid composition according to claim 1, comprising at least the steps consisting of:
(a) having available a fatty phase, a phase derived from a saponification reaction and/or a phase comprising at least one synthetic detergent (or surfactant) (i.e. SYNDET phase), in liquid or semi-liquid form;
(b) passing the phase of step (a) through an extrusion device equipped with a die (or extrusion grating) with a shape suitable for forming a solid bar, and
(c) cutting the solid bar obtained in step (b), whereby a the solid composition is obtained.

13. A non-therapeutic method for cosmetic treatment for the hygiene or the beautifying of a keratin material, comprising at least one step of applying to said keratin material at least the solid composition according to claim 1.
